(19) United States
(12) Patent Application Publication
(10) Pub. No.: US 2008/0200790 A1
(43) Pub. Date: Aug. 21, 2008

(54) Apparatus For Measuring Blood Sugar and Apparatus For Monitoring Blood Sugar Comprising the Same

(75) Inventors: Jong Chel Kim, Seoul (KR); Seung Wan Kim, Seoul (KR)

Correspondence Address: BRADLEY ARANT ROSE & WHITE LLP, 200 CLINTON AVE. WEST, SUITE 900, HUNTSVILLE, AL, 35801, US

(73) Assignee: Ricci Microwave Corp., Seoul (KR)

(21) Appl. No.: 11/994,397

(22) PCT Filed: Dec. 29, 2006
(86) PCT No.: PCT/KR06/05873

§ 371 Date: Dec. 31, 2007

(30) Foreign Application Priority Data
Dec. 30, 2005 (KR) 10-2005-0134655

(51) Int. Cl. A61B 5/145; G06Q 50/00

(52) U.S. Cl. 600/365; 705/2

(57) ABSTRACT

There is provided an apparatus for measuring a blood sugar by using a microwave without withdrawing any blood while enhancing the reliability of measurement. The apparatus for measuring the blood sugar according to the present invention has a main body having a measurement surface configured to contact a measurement portion of a user, a probe part having a contact member exposed on the measurement surface so as to be in contact with the measurement portion, the probe part further having a probe disposed under the contact member for irradiating and receiving a microwave, a blood sugar measuring unit for supplying the microwave to the probe and measuring a blood sugar value from the received microwave, and a securing unit mounted on the main body for securing the measurement portion to the measurement surface.

### TECHNICAL FIELD

The present invention generally relates to an apparatus for measuring blood sugar, and more particularly to an apparatus for reliably measuring the blood sugar of a user without taking any blood by using a microwave. Further, the present invention also relates to an apparatus for monitoring the blood sugar, which comprises the above apparatus for measuring the blood sugar.

### BACKGROUND ART

Generally, a patient suffering from diabetes must always examine his/her own blood sugar value and be treated when the blood sugar value is too high or too low. Various types of blood sugar measuring apparatus have been introduced in the art so that the patient suffering from diabetes can periodically examine his/her own blood sugar value.

For example, there is a blood sugar measuring instrument or a test paper for measuring the blood sugar from blood taken from the patient. However, since this involves withdrawing blood from the patient, problems exist in that the conditions for withdrawing blood may be unsanitary and the patient is continuously subjected to pain.

As an alternative to the above, there has recently been introduced a technique, wherein an electric wave is radiated into the patient's body and the blood sugar value is then measured from the reflected electric wave. This is somewhat advantageous since the blood sugar of the patient can be measured quantitatively without withdrawing any blood. However, such blood sugar measurement is highly unreliable due to reduced accuracy and reproducibility, while being prone to errors.

### DISCLOSURE OF INVENTION

### Technical Problem

Therefore, it is an object of the present invention to provide an apparatus for measuring blood sugar without taking any blood by using a microwave.

It is a further object of the present invention to provide such an apparatus capable of measuring blood sugar with high accuracy and reproducibility.

It is another object of the present invention to provide an apparatus for monitoring blood sugar, which comprises the above measuring apparatus, and is capable of communicating with a hospital staff located at a remote distance.

### Technical Solution

In order to achieve the above and other objects, the present invention provides an apparatus for measuring blood sugar, comprising: a main body having a measurement surface configured to contact a measurement portion of a user; a probe part having a contact member disposed on the measurement surface so as to be in contact with the measurement portion, the probe part further having a probe disposed under the contact member for irradiating and receiving a microwave; means for measuring blood sugar for supplying the microwave to the probe and measuring a blood sugar value from the received microwave; and means mounted on the main body for securing the measurement portion to the measurement surface.

The probe part has a dielectric resonator, to which the microwave from the measuring means is supplied. The probe is disposed on the dielectric resonator. The probe part also has a guide member disposed on the dielectric resonator so as to surround the probe. The guide member is fabricated from metal.

The measuring means includes: a microwave-generating part for generating a microwave having a center frequency and supplying the microwave to the dielectric resonator; a microwave-detecting part for generating a voltage signal from the received microwave; a memory part for storing blood sugar values corresponding to the voltage signals of the received microwaves; and a control part for comparing the voltage signal detected by the microwave-detecting part with the voltage signal stored within the memory part and outputting the blood sugar value corresponding to the detected voltage signal.

The microwave-generating part generates a centimeterwave having a fixed center frequency.

The securing means includes a pressing plate for pressing the measurement portion toward the measurement surface. The securing means is configured to surround the measurement surface and is consisted of an elastic material so as to be expandable according to the measurement portion. The surface of the securing means, which faces the measurement portion, is coated with a conductive substance.

Further, the blood sugar-measuring apparatus further comprises an image-recognizing means disposed on the measurement surface for interacting with the control part and identifying the measurement portion of the user.

Also, the measuring apparatus of the present invention is configured so that the image-recognizing means reads an image of user's dermatoglyphics, wherein said image is stored in the memory part. Further, the control part performs the identification of a user and the measurement of blood sugar by using the stored image.

The displaying means includes: an input part, to which blood sugar data are inputted from the control part; a display part for displaying the inputted blood sugar data; and an output part for externally outputting the blood sugar data.

According to another aspect of the present invention, there is provided an apparatus for monitoring blood sugar, comprising: the above-described apparatus for measuring blood sugar; a computer server for a user, which is connected to the measuring apparatus, and to which the measured blood sugar value is inputted; a computer server for the hospital staff, which is connected to the computer server for the user via a two-way communication network, and to which the blood sugar value is transmitted; and a terminal connected to the computer server for the hospital staff and being configured so that the transmitted blood sugar value is identified by the hospital staff.

### Advantageous Effects

The present invention may provide the following advantages.

First, since the blood sugar is measured without withdrawing any blood by using a microwave, there is no need to worry about sanitation problems or pain caused to the user.

Second, since the electromagnetic field of a microwave is focused on a probe by a guide member, the microwave is irradiated with strong intensity. This enhances the accuracy and reliability of a blood sugar measurement.

Third, since a measurement surface and a measurement portion are fixedly secured to each other, the accuracy of measurement is increased while decreasing measurement errors.

Fourth, since a conductive substance is provided on a portion of securing means (except for a portion in which the microwave is irradiated and received), the efficiency of irradiating and receiving the microwave is enhanced while decreasing the noise.

Fifth, a first measurement portion is determined by an image-recognizing sensor and the measurement can be initiated only when an exact measurement portion is positioned by comparing with the first determined measurement portion at the next measurement. Thus, the reproducibility of measurement is increased.

Sixth, since the measurement surface and the measurement portion can be secured without any relative movement therebetween due to the securing means, a continuous measurement is possible while increasing the convenience of measurement.

Seventh, since the result of measurement is transmitted via a two-way communication network, a prescription for measurement can be transmitted in real time from the hospital staff to the user.

### BRIEF DESCRIPTION OF THE DRAWINGS

FIG. 1 is a perspective view of an apparatus for measuring blood sugar constructed in accordance with the first embodiment of the present invention.

FIG. 2 is a perspective view showing a probe part.

FIG. 3 is a sectional view showing the probe part.

FIG. 4 is a block diagram showing the constitution of the blood sugar-measuring apparatus shown in FIG. 1.

FIG. 5 is a perspective view of an apparatus for measuring blood sugar constructed in accordance with the second embodiment of the present invention.

FIG. 6 is a perspective view of an apparatus for measuring blood sugar constructed in accordance with the third embodiment of the present invention.

FIG. 7 is a perspective view of an apparatus for measuring blood sugar constructed in accordance with the fourth embodiment of the present invention.

FIG. 8 is a schematic diagram showing the constitution of an apparatus for monitoring blood sugar according to another aspect of the present invention.

FIG. 9 is a graph showing a blood sugar value according to an output value of a resonant frequency.

### BEST MODE FOR CARRYING OUT THE INVENTION

An apparatus for measuring blood sugar and an apparatus for monitoring blood sugar comprising the same will now be described in detail with reference to the accompanying drawings.

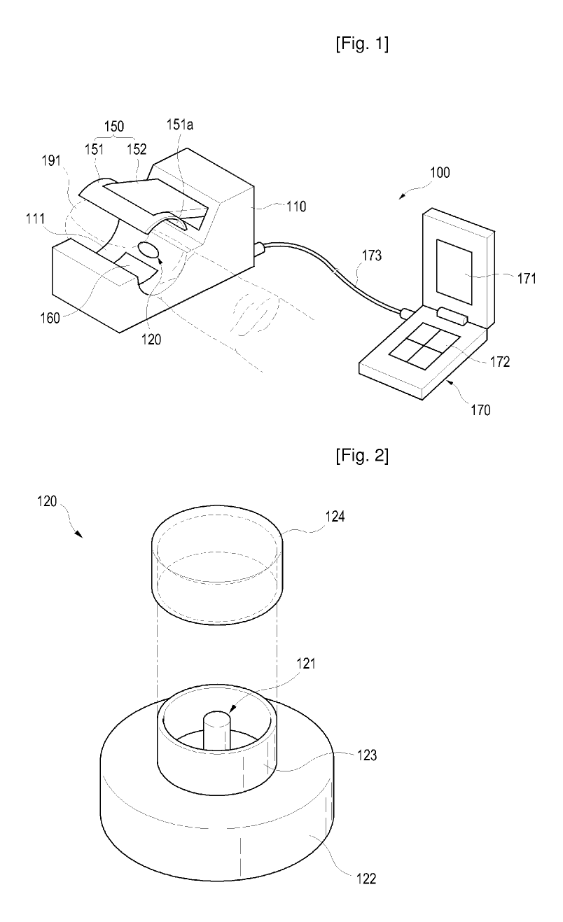

FIG. 1 is a perspective view of a blood sugar-measuring apparatus 100, which is constructed in accordance with the first embodiment of the present invention.

The blood sugar-measuring apparatus 100 of the present invention is configured to measure a blood sugar value of a user without withdrawing any blood due to the use of a microwave. Referring to FIG. 1, the blood sugar-measuring apparatus 100, which is constructed in accordance with the first embodiment of the present invention, comprises a main body 110, wherein a portion of a user's body (e.g., fingertip 191) is placed and the blood sugar is measured. Such an apparatus 100 also comprises securing means 150 for securing the fingertip 191 with respect to the main body 110. The measured blood sugar value can be displayed by displaying means 170, which is connected to the main body 110 via a cable 173.

The blood sugar-measuring apparatus 100 contacts a measurement portion of the user and measures the blood sugar value of a blood stream, which passes through blood vessels within the user's body, by using a microwave. In this embodiment, the measurement portion is a fingertip 191. Further, a blood sugar measurement is performed when the fingertip 191 is placed on a measurement surface 111, which is formed on an upper side of the main body 110.

The measurement surface 111 may be formed on the upper side of the main body 110 either as a flat surface or a concave surface, as shown in FIG. 1. This is so that the fingertip 191 may be rested thereon. On the measurement surface 111, there is provided a probe part 120, which irradiates and receives a microwave while contacting the measurement portion of the user.

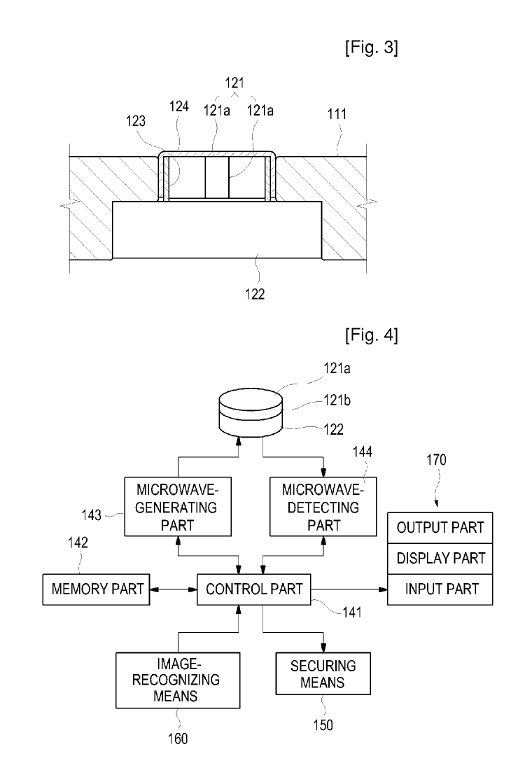

FIG. 2 is a schematic perspective view showing the probe part 120, whereas FIG. 3 is a sectional view showing the probe part 120. Referring to FIGS. 2 and 3, the probe part 120 comprises the following: a probe 121 for irradiating and receiving a microwave; a dielectric resonator 122; a guide member 123 disposed around the probe 121 for focusing an electromagnetic field of the microwave on the probe 121; and a contact member 124 for covering the probe 121 and the guide member 123, wherein the contact member is configured to be in direct contact with the measurement portion of the user. The probe 121 includes an antenna portion 121b, which is mounted on the dielectric resonator 122, and through which the microwave is irradiated and received. The probe 121 also includes a contact surface 121a, which is configured to contact the measurement portion of the user via the contact member 124.

The guide member 123 is made from a metallic material and has a cylindrical shape. The guide member 123 is positioned so that the probe 121 is centered therein. Thus, the electromagnetic field of the microwave irradiated from the probe 121 can be focused on the probe 121 without being irradiated outside the guide member 123. The contact member 124 is placed so as to be in close contact with an upper surface of the probe 121 and is further fitted to the guide member 123. The contact member has a dielectric constant of 1˜10 and a thickness of 0.5˜5 mm. The contact member may be made from glass or synthetic resins.

Since the contact member 124 is located on the upper surface of the probe 121, the probe 124 can contact the measurement portion via the contact member 124. Therefore, the probe 121 detects a variation of glucose by perturbation action with respect to blood sugar (glucose) within the measurement portion in a near-field environment when it is not in direct contact with the measurement portion. While the variation of blood sugar within the measurement portion is very small, the microwave irradiated from the probe 121 is directed to the measurement portion with strong intensity. This is because the electromagnetic field of the microwave is focused on the probe 121 by the guide member 123, as described above. Accordingly, any influences or impacts from peripheral noises can be minimized while enhancing the accuracy of measurement.

Referring once again to FIG. 1, on a central portion of the measurement surface 111, there is provided an image-recognizing sensor (e.g., a fingerprint sensor 160), which is capable of reading the dermatoglyphics of the user, as image-recognizing means for identifying a specific user. The fingerprint sensor 160 is exposed on the measurement surface 111 such that it can contact the user's fingertip 191.

The blood sugar value of the user, which is measured by the probe part 120, is displayed to the user by means of displaying means 170. The displaying means 170 is a display device including a display window 171, on which the blood sugar value can be displayed as graphs, numerals or letters. The displaying means 170 also includes operating keys or switches 172 for operating the blood sugar-measuring apparatus 100.

As the securing means for securing the measurement portion 191 with respect to the main body 110 and preventing a positional change therebetween, a pressing plate 151 and a member 152 for driving the pressing plate are provided in the main body 110. The pressing plate 151 is made from flexible metal or synthetic resins. The pressing plate 151 has an arcuate shape so that it can uniformly press down on the fingertip 191. The drive member 152, which moves the pressing plate 151, has an arm shape. The drive member 152 is configured to be rotated with respect to the main body 110 and press the pressing plate toward the fingertip 191. The drive member 152 may be operated by using an electrical motor or an electromagnet. The drive member 152 is not limited to only the arm shape shown in FIG. 1. The drive member may be configured in any shape so long as the pressing plate 151 can be lowered down from the top. It is preferred that an extent, to which the fingertip 191 is pressed by the drive member 152, is determined. This is so that the user does not feel any pain and the positional change between the measurement surface 111 and the fingertip 191 does not take place. As a simpler configuration, the pressing plate 151 and the drive member 152 may be configured in a manner so that the fingertip 191 is pressed by a spring.

When measuring the blood sugar by using a microwave, the reliability of the measured blood sugar values goes down in case the measurement portion moves or the same measurement portion is not placed. However, the fingertip 191 is secured with respect to the measurement surface 111 by the pressing plate 151, thereby allowing the microwave to be irradiated and received for a fixed portion.

Preferably, a portion of the measurement surface 111 (except the probe part 120 and the fingerprint sensor 160) is coated with a conductive substance or includes a conductive substance. Further, in case of a synthetic resins-made pressing plate 151, a surface 191a of the pressing plate facing the fingertip 191 is coated with a conductive substance.

The blood sugar measurement is carried out in a manner so that the microwave is irradiated via the probe part 120 and the reflected microwave is received via the probe part 120. In such a case, it is preferred that the microwave is irradiated into the fingertip 191. However, some of the microwave escapes outwardly due to the radiation and does not return or returns after interacting with an unwanted object, thereby causing a noise. However, as described above, when the conductive substance is coated on the portion of the measurement surface 111 (except the probe part 120 and the fingerprint sensor 160) and the surface 151a of the pressing plate 151 facing the fingertip 191, the microwave fails to penetrate the portion having such conductive substance. Consequently, since the microwave is irradiated into and received from the measurement portion via the probe part 120 at the portion surrounding the measurement portion (i.e., the measurement surface 111 and the inner surface 151a of the pressing plate), the accuracy of the blood sugar measurement is enhanced.

FIG. 4 is a block diagram showing the constitution of the blood sugar-measuring apparatus 100 shown in FIG. 1. Various elements shown in FIG. 4 may be provided within the main body 110. Alternatively, some of the elements may be provided outside the main body 110, if necessary.

A microwave-generating part 143 generates the microwave and the adjustment of its frequency is carried out by a control part 141. More specifically, the microwave-generating part 143 generates a centimeterwave having a center frequency, which is selected between 10 [symbol] to 20 [symbol] and then fixed. The generated centimeterwave enters the dielectric resonator 122.

The dielectric resonator 122 forms a propagation mode of the transmitted centimeterwave into a transverse electric mode, a transverse magnetic mode or a transverse electromagnetic mode. The transmitted centimeterwave is changed into a centimeterwave having a resonant frequency of 2.048 [symbol] via the dielectric resonator 122 and is then irradiated into the measurement portion 191 via the antenna portion 121b and the contact surface 121a. The irradiated centimeterwave returns back to the probe 121 after being perturbed with glucose within a blood stream passing through the blood vessel in the fingertip 191 (measurement portion). Thereafter, the centimeterwave, having returned to the probe 121, is detected by a microwave-detecting part 144.

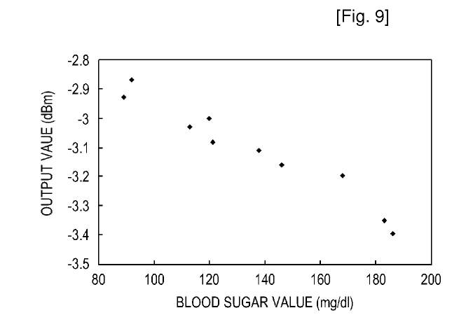

More specifically, the centimeterwave, which is generated by the microwave-generating part 143 and has a center frequency of 2.048 [symbol] has a maximum amplitude S₁₁ and S₁₂ and a determined phase value due to an impedance matching caused by tuning. These values are changed due to the perturbation from glucose within the measurement portion. The changed values are displayed after being changed into voltage, as shown in FIG. 9. In such a case, the perturbation occurs between the center frequency of 2.048 [symbol] and the glucose. This is because when the electromagnetic field of the center frequency interacts with the glucose, the properties of the microwave, e.g., a center frequency, the amplitude S11 and S21 of the center frequency and the phase value of the center frequency are influenced by a dielectric constant of the glucose and are thus changed.

For the above description, the following can be described as to how a dielectric constant change of blood sugar (glucose) in NaCl solution influences a change in reflectivity S₁₁.

First, the reflectivity S₁₁ for the dielectric constant can be shown in the following Equation 1 by assuming an impedance matching between the probe and the resonant sensor as well as using a transmission line theory.

$${S}_{11} = 20 log \vert \frac{{Z}^{R} - {Z}_{0}}{{Z}^{R} + {Z}_{0}} \vert , {S}_{11}^{2} + {S}_{21}^{2} = 1 \qquad [ Equation 1 ]$$

wherein Z₀ shows an effective impedance of the probe and is matched to 50Ω. Z^{R} is a real part of a complex impedance of the glucose solution, which is in a cylindrical cell substrate and can be shown as follows.

$${Z}^{R} = Re [ \frac{{Z}_{a}}{\sqrt{ɛ}} \times \frac{( {Z}_{a} / \sqrt{{ɛ}_{s}} ) + j ( {Z}_{a} / \sqrt{ɛ} ) tan ( {k}_{a} \sqrt{ɛ} ( v / s ) )}{( {Z}_{a} / \sqrt{ɛ} ) + j ( {Z}_{a} / \sqrt{{ɛ}_{s}} ) tan ( {k}_{a} \sqrt{ɛ} ( v / s ) )} ] \qquad [ Equation 2 ]$$

wherein Zₐ is an impedance of air (377Ω), kₐ is a wave number of air (84 m⁻¹ at 4 [symbol] ), ε is a dielectric constant of the cylindrical glass cell substrate, vis a volume of the solution, and s is a surface area of the solution (25 mm²). The dependence of the dielectric constant with respect to the concentration of the solute glucose is linear. This is shown as a molar increment. In such a case, the dielectric constant E of the NaCl solution can be shown in the form of a complex number as the following Equation 3.

ε=(ε₀′+cγ′)−j(ε₀″+cγ″) [Equation 3]

wherein ε₀ is a dielectric constant of water (at 26° C. and 4 [symbol] , ε′ is 75.3 and ε″ is 14.58), c is a concentration of the glucose solution, and γ is an increment of the dielectric constant when the concentration of the solution is raised by one unit (in case of NaCl, γ′ is 0.0695(mg/ml)⁻¹ and γ″ is 0.0019(mg/ml)⁻¹; and in case of glucose, γ′ is 0.0577(mg/ml)⁻¹ and γ″ is 0.0015(mg/ml)⁻¹). Therefore, the real part of the complex impedance of the glucose solution, which is in the glass substrate, can be calculated as the following Equation 4.

$${Z}^{R} = \frac{{Z}_{a}}{\sqrt{{ɛ}_{s}}} \times \frac{1 + {[ tan ( {k}_{a} ( v / s ) \sqrt{{ɛ}_{0}^{′} + c {δ}^{′}} ) ]}^{2}}{1 + {[ ( {ɛ}_{0}^{′} + c {δ}^{′} ) / {ɛ}_{s} ] [ tan ( {k}_{a} ( v / s ) \sqrt{{ɛ}_{0}^{′} + c {δ}^{′}} ) ]}^{2} } \qquad [ Equation 4 ]$$

As can be seen from the Equation 4, the ionic dielectric constant of the solution and the intensity of the reflectivity S₁₁ are increased with the increase of the concentration of the glucose.

For example, when a centimeterwave, which has a S₂₁ amplitude of −1.9496 [symbol] at a center frequency of 2.048 [symbol] , is brought into contact with the glucose within the body via the probe, the resonant frequency is shifted by the dielectric constant of the glucose and S₁₁ is changed such that the amplitude is lowered to −1.9541 [symbol] at 2.048 [symbol] (15.6 [symbol] ). It is possible to display the blood sugar value by changing this into a voltage.

Consequently, in the blood containing the glucose, the dielectric constant changes according to the amount of the contained glucose. Therefore, the variation of the blood sugar in the blood can be measured by means of the variation of the dielectric constant. Such variation of the dielectric constant can be obtained by analyzing the variation of the resonance point of the returning centimeterwave after reflection. The resonant frequency reacts to the glucose within the blood and returns to the probe with its resonance point shifted. The microwave-detecting part 144 detects a voltage signal from the power signal type centimeterwave having a voltage and a current and transmits it to the control part 141.

The voltage signal detected by the microwave-detecting part 144 is sent to the control part 141 and is processed at the control part. The control part 141 converts the voltage signal transmitted from the microwave-detecting part 144 through an A/D converter and the like. It then outputs the measured blood sugar value after interacting with a memory part 141. In the memory part 141, the blood sugar values are stored, which correspond to the detected voltage signals, as data for a criterion of comparison. The control part 141 compares the voltage signal detected by the microwave-detecting part 144 with the voltage signal stored in the memory part 141 and outputs the blood sugar value corresponding to the detected voltage signal. One example of the data stored in the memory part 141 is shown as a graph of FIG. 9. FIG. 9 shows the blood sugar values according to the output values of the returning centimeterwave.

The data of the blood sugar value, which is outputted from the control part 141, is transmitted to the display device 170. The display device 170 includes an input part, to which the data of the measured blood sugar value is inputted. The display device 170 also includes a display part for displaying the inputted data of the blood sugar value on the display window 171 as graphs, numerals, letters and the like. Further, the display device 170 may further include an output part for transmitting the data of the measured blood sugar value outside the display device. The output part, which transmits the data of the measured blood sugar value outside the apparatus 100, may be provided not in the display device 170 but in the main body 110 (e.g., the control part 141).

The blood sugar-measuring apparatus 100 of the present invention facilitates the maintenance and protection of an individual blood sugar value of the user and can identify the specific user for a customized blood sugar examination. To this end, the blood sugar-measuring apparatus 100 comprises image-recognizing means. In this embodiment, the image-recognizing means is the fingerprint sensor 160, which is exposed on the measurement surface 111.

The fingerprint sensor 160 identifies the specific user while interacting with the control part 141. Further, the control part 141 operates the components 143, 121, 144 relating to the blood sugar measurement and the securing means 150 for securing the measurement portion 191 to the main body 111 (e.g., the pressing plate 151 and the drive member 152) in response to the sensed result from the fingerprint sensor 160. For example, when the fingerprint sensor 160 identifies the fingerprint of the specific user, the control part 141 operates the securing means 150. This is so that it can secure the fingertip 191 (the measurement portion) with respect to the main body 110 and then measures the blood sugar value by interacting with the microwave-generating part 143, the microwave-detecting part 144 and the memory part 141. The measured blood sugar value is displayed to the specific user by the display device 170.

Further, when measuring the blood sugar by using a microwave, if the same measurement portion of the user is placed on the measurement surface 111 in every measurement, then there is a reproducibility of measurement. In order to identify whether the same measurement portion is placed on the measurement surface 111 in every measurement or not, the control part 141 stores a first fingerprint image of the specific user obtained by the fingerprint sensor 160 to the memory part 141 and then uses it as a criterion for comparison in the next measurement.

More specifically, when the specific user uses the blood sugar-measuring apparatus 100 of this embodiment, the user first places his/her own fingertip 191 on the measurement surface 111 and stores the recognized fingerprint by the fingerprint sensor 160 to the memory part 141. In the next measurement, if the user places his/her fingertip 191 on the fingerprint sensor 160, then the control part 141 interacts with the fingerprint sensor 160 and the memory part 141. It then emits a sound or informs the user of being in position of the measurement portion through the display window. Thereafter, the control part 141 operates the securing means 150 so that the fingertip 191 is secured to the main body 110 and then carries out the blood sugar measurement.

When the display device 170 is configured to transmit a data to the control part 141, the above-discussed recognition and storage of the first fingerprint may be conducted by the user through the keys or switches 172 of the display device 170. Further, when the control part 141 has a reset function, the recognition and storage of the first fingerprint can be newly renewed. As such, the blood sugar-measuring apparatus 100, which only the specific user uses, can be provided. On the other hand, the blood sugar-measuring apparatus 100, which a plurality of users can use according to the capacity of the memory part 142 and the performance of the control part 141, can be provided.

In the blood sugar-measuring apparatus 100 of the present invention, a plurality of probes 121 may be provided. Further, a plurality of microwave-detecting parts 144 may also be provided accordingly. While it is shown that a single probe part 120 appears on the measurement surface 111 in FIG. 1, a plurality of probe parts 120 may appear on the measurement surface 111. In such a case, the control part 141 statistically processes a plurality of data from numerous microwave-detecting parts 144 and determines more accurate blood sugar value, thereby decreasing the measurement errors and enhancing the accuracy of measurement.

Further, the display device 170 shown in FIG. 1 is configured for the blood sugar-measuring apparatus 100. However, the display device 170 is certainly not limited thereto. A mobile phone, a PDA, a notebook computer, a desktop computer and the like, into which an appropriate software are programmed, may be employed as the display device.

Also, while the measurement portion in this embodiment is the fingertip 191, when the main body 110 and the pressing plate 151 are suitably sized, the measurement portion may be a middle part of a finger or even the wrist.

Additionally, if the display device 170 is configured to transmit a data to the control part 141, instead of a user-identification function of the fingerprint sensor 160, the blood sugar-measuring apparatus 100 may be configured such that the user inputs an ID and a password through the operating keys or switches 172. In such a case, the inputs are transmitted to the control part 141, wherein the control part 141 starts the operation of the apparatus 100.

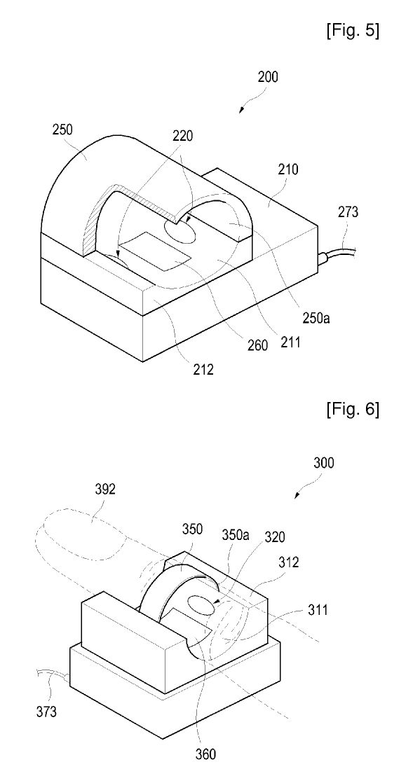

FIG. 5 shows a blood sugar-measuring apparatus 200 constructed in accordance with the second embodiment. The blood sugar-measuring apparatus 200 of this embodiment has the same configuration as the blood sugar-measuring apparatus 100 of the first embodiment (except that a thimble-shaped securing member 200 is provided as securing means for securing the measurement portion to the main body 210 and the securing member 250 is not operated by the control part (not shown) provided in the main body 210).

Preferably, the thimble-shaped securing member 250 is made from an elastic material so that it can expand when the measurement portion of the user (e.g., fingertip) is inserted thereto. For example, the securing member 250 may be made from a material such as rubber, silicone and the like. An extent, to which the fingertip 191 is pressed by the expansion of the securing member 250, is determined so that the user does not feel any pain. When the fingertip of the user is inserted, the securing member 250 presses the fingertip (the measurement portion) toward the measurement surface 211 while expanding. Therefore, the measurement portion and the measurement surface 211 can be secured without any positional change.

The measurement surface 211, which is in contact with the measurement portion, may be in the form of a flat surface or a concave groove formed in a seat 212, which is provided on the main body 210, as shown in FIG. 5. The seat, which has the measurement surface 211 thereon and on which the fingertip is placed, is made from an elastic material such as rubber or silicone. The microwave is irradiated and received by the probe part 220. Identification of the specific user or verification of an exact position regarding the measurement portion is carried out by the fingerprint sensor 260. Further, as described above, the portion of the measurement surface 211, on which the probe part 220 and the fingerprint sensor 260 are not exposed, and the inner surface 250a of the securing means 250 may be coated with the conductive substance. Alternatively, however, they may comprise the conductive substance in order to enhance the efficiency of irradiating and receiving the microwave. The measured blood sugar value is transmitted to the display device (not shown) via the cable 273 and is displayed to the user.

FIG. 6 shows a blood sugar-measuring apparatus 300 constructed in accordance with the third embodiment. The blood sugar-measuring apparatus 300 of this embodiment has the same configuration as the blood sugar-measuring apparatus 200 of the second embodiment (except that the measurement portion is a middle part of a finger; a ring-shaped securing member 350 is provided as securing means for securing the measurement portion to the measurement surface 311 of the main body 310; and the image-recognizing means is configured to read the dermatoglyphics of the middle part of a finger).

The ring-shaped securing member 350 is made from an elastic material so that it can expand when the measurement portion of the user (e.g., the middle part of a finger) is inserted thereto. For example, the securing member 350 may be made from a material such as rubber, silicone and the like. Its expansion extent is determined so that the user does not feel any pain in his/her middle part of the finger. When the middle part of the user's finger is inserted, the securing member 350 presses the middle part of the finger (the measurement portion) toward the measurement surface 311 while expanding. Thus, the measurement portion and the measurement surface 311 can be secured.

The measurement surface 311, which is in contact with the measurement portion, may be formed as a flat surface or a concave groove formed in a seat 312, which is provided on the main body 310, as shown in FIG. 6. The seat, which has the measurement surface 311 thereon and on which the middle part of a finger is placed, is made from the same material as the securing member 350. The microwave is irradiated and received by the probe part 320. Identification of the specific user or verification of an exact position regarding the measurement portion is carried out by the image-recognizing means 360. The image-recognizing means 360 is configured to read the dermatoglyphics or the skin wrinkle pattern of the middle part of the finger. For example, a fingerprint sensor or any other image sensor may be employed as the image-recognizing means. Further, as described above, the dermatoglyphics or the skin wrinkle pattern of the middle part of the finger may be stored in the memory part (not shown) inside the apparatus 300 by the user when the apparatus 300 is first used. At every subsequent measurement, the control part (not shown) may be configured to identify the specific user and to initiate the measurement when the middle part of the finger is positioned by using such stored pattern. Thus, the reproducibility of measurement and the reliability of measurement results can be enhanced.

Further, as described above, the portion of the measurement surface 311, on which the probe part 320 and the image-recognizing means 360 are not exposed, and the inner surface 350a of the securing member 350 may be coated with the conductive substance. Alternatively, however, they may comprise the conductive substance in order to enhance the efficiency of irradiating and receiving the microwave. The measured blood sugar value is transmitted to the display device (not shown) via the cable 373 and is then displayed to the user.

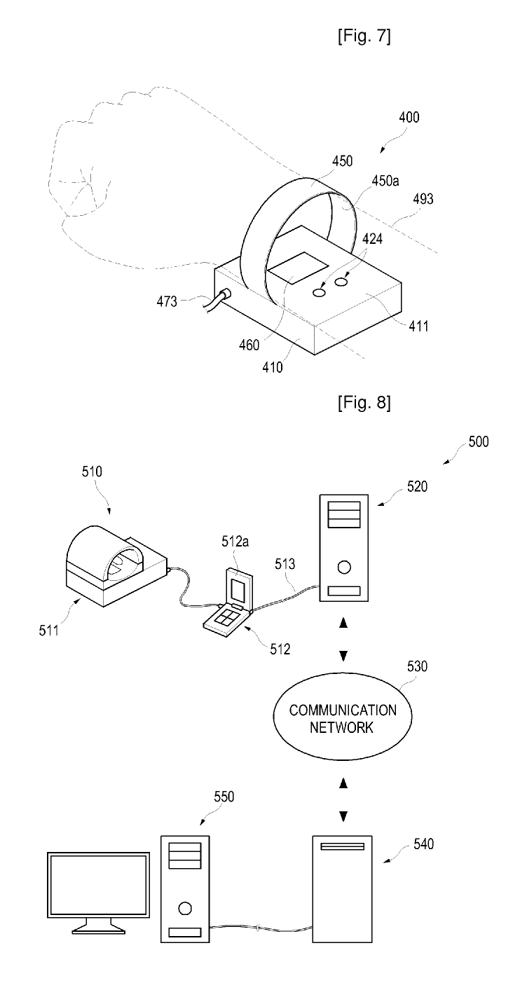

FIG. 7 shows a blood sugar-measuring apparatus 400 constructed in accordance with the fourth embodiment. The blood sugar-measuring apparatus 400 of this embodiment has the same configuration as the blood sugar-measuring apparatus 300 of the third embodiment (except that the measurement portion is the wrist and a wristband-shaped securing member 450 is provided as securing means for securing the measurement portion to the main body 410).

The wristband-shaped securing member 450 is made from an elastic material so that it can expand when the wrist of the user is inserted thereto. For example, the securing member 450 may be made from a single body consisting of rubber, silicone and the like, or may be made from a band-shaped fabric with a rubber band incorporated therein. Its expansion extent is determined so that the user does not feel any pain in his/her wrist and the wrist and the measurement surface 411 are moved to each other. When the wrist of the user is inserted, the securing member 450 presses the wrist toward the measurement surface 411 while expanding. Therefore, the measurement portion and the measurement surface 411 can be secured.

The measurement surface 411 may be formed as a flat surface (shown in FIG. 7) or a concave groove formed in a seat made from rubber or silicone, similar to the second or third embodiment. The microwave is irradiated and received by the probe part 420. Identification of the specific user or verification of an exact position regarding the measurement portion is carried out by the image-recognizing means 460. The image-recognizing means 460 is configured to read the dermatoglyphics or the skin wrinkle pattern of the user's wrist. For example, a fingerprint sensor or any other image sensor may be employed as the image-recognizing means. Further, as described above, the dermatoglyphics or the skin wrinkle pattern of the wrist may be stored in the memory part (not shown) inside the apparatus 400 by the user when the apparatus 400 is first used. As such, at every subsequent measurement, the apparatus 400 may be configured to identify the specific user and operate when the wrist is placed in position. Therefore, the reproducibility of measurement and the reliability of measurement results can be enhanced.

Further, as described above, the portion of the measurement surface 411, on which the probe part 420 and the image-recognizing means 460 are not exposed, and the inner surface 450a of the securing member 450 may be coated with the conductive substance. Alternatively, however, they may comprise the conductive substance in order to enhance the efficiency of irradiating and receiving the microwave. The measured blood sugar value is transmitted to the display device (not shown) via the cable 473 and is then displayed to the user.

FIG. 8 schematically shows an apparatus for monitoring the blood sugar 500 according to another aspect of the present invention. The apparatus for monitoring the blood sugar 500 is configured such that the hospital staff located at a remote distance can monitor the measured blood sugar values, which are measured by the above blood sugar-measuring apparatus.

The measured blood sugar values, which are measured by the blood sugar-measuring apparatus 510 in accordance with one embodiment of the present invention, are transmitted to a computer server for a user 520 through the display device 512 via the cable 513. When the display device 512 is provided as shown, the measured blood sugar values may be displayed on the display window 512a. Further, when the output part, which is shown in FIG. 4, has a wireless transmission function and the server 520 also has a wireless reception function, the blood sugar values measured by the blood sugar-measuring apparatus 510 may be wirelessly transmitted to the server 520. Also, the display device 512 may be omitted and the main body 511 can be directly connected to the server 520.

The transmitted blood sugar values are transmitted from the computer server for a user 520 via a communication network 530 to a computer server for the hospital staff 540. The computer server for the hospital staff 540 sends the transmitted blood sugar values to a computer terminal 550, to which the hospital staff can have access, via a local area network or an intranet. Therefore, the hospital staff in charge can monitor the blood sugar values of the user in real time. The communication network 530 may be a wired or wireless internet network.

Since the wired or wireless internet network 530 allows a two-way communication, the hospital staff in charge monitors the blood sugar values of the user displayed on their own terminal 550 and can thus prescribe accordingly. Then, the data of such prescription is transmitted once again to the computer server for the hospital staff 540, the wired or wireless internet network 530 and the computer server for a user 520. The prescription, which is transmitted to the computer server for a user 520, is then transmitted to the display device 512 via the cable 513. Then, the result of prescription can be displayed on the display window 512a in real time.

Further, since the fingerprint sensor is employed as the image-recognizing means in the blood sugar-measuring apparatus 510 shown in FIG. 8, as described above, the hospital staff in charge can identify the specific user by fingerprint recognition. Also, in case the blood sugar-measuring apparatus operates when the user inputs his/her own ID and password, the ID data may be transmitted to the hospital staff in charge who may then identify the specific user.

The blood sugar-measuring apparatus, which is describe above, may be supplied to the user together with a user's manual as a product for measuring the blood sugar. For example, a product for measuring the blood sugar (including an apparatus for measuring the blood sugar according to the present invention) and a user's manual (for explaining a measurement procedure, a measurement time, how to use the apparatus and the like) can be supplied to the user. In such a case, the display device may be specially designed so as to display only the blood sugar values. Alternatively, it may be a mobile phone, a PDA, a notebook computer and the like, into which an appropriate software is programmed, if necessary.

While the present invention has been particularly shown and described with reference to exemplary embodiments thereof, it will be understood by those of ordinary skill in the art that various changes in form and details may be made therein without departing from the spirit and scope of the present invention as defined by the following claims.

### INDUSTRIAL APPLICABILITY

According to the present invention, there is provided a blood sugar-measuring apparatus, wherein blood sugar can be simply measured without withdrawing any blood by using a microwave while the accuracy and reproducibility of measurement are enhanced. Further, there is provided an apparatus for monitoring the blood sugar, which comprises the above blood sugar-measuring apparatus and is capable of communicating with a hospital staff located at a remote distance.

1. An apparatus for measuring a blood sugar, comprising: a main body having a measurement surface configured to contact a measurement portion of a user; a probe part having a contact member exposed on the measurement surface so as to be in contact with the measurement portion, the probe part further having a probe disposed under the contact member for irradiating and receiving a microwave; means for measuring the blood sugar for supplying the microwave to the probe and measuring a blood sugar value from the received microwave; and means mounted on the main body for securing the measurement portion to the measurement surface.

2-13. (canceled)

14. An apparatus for monitoring a blood sugar, comprising: an apparatus for measuring the blood sugar according to claim 1; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

15. The apparatus of claim 14, wherein the apparatus is configured so that a data of prescription from the hospital staff is transmitted to the blood sugar-measuring apparatus via the two-way communication network.

16. The apparatus of claim 1, wherein the probe part has a dielectric resonator, to which the microwave from the measuring means is supplied, and the probe is disposed on the dielectric resonator, and wherein the probe part further has a guide member disposed on the dielectric resonator so as to surround the probe and comprising a metal.

17. An apparatus for monitoring a blood sugar, comprising: an apparatus for measuring the blood sugar according to claim 16; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

18. The apparatus of claim 17, wherein the apparatus is configured so that a data of prescription from the hospital staff is transmitted to the blood sugar-measuring apparatus via the two-way communication network.

19. The apparatus of claim 16, wherein the measuring means includes: a microwave-generating part for generating the microwave having a center frequency and supplying the microwave to the dielectric resonator; a microwave-detecting part for generating a voltage signal from the received microwave; a memory part for storing blood sugar values corresponding to the voltage signals of the received microwaves; and a control part for comparing the voltage signal detected by the microwave-detecting part with the voltage signal stored within the memory part and outputting the blood sugar value corresponding to the detected voltage signal.

20. An apparatus for monitoring a blood sugar, comprising: an apparatus for measuring the blood sugar according to claim 19; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

21. The apparatus of claim 20, wherein the apparatus is configured so that a data of prescription from the hospital staff is transmitted to the blood sugar-measuring apparatus via the two-way communication network.

22. The apparatus of claim 19, wherein the microwave generating part generates a centimeter wave having a fixed center frequency.

23. An apparatus for monitoring a blood sugar, comprising: an apparatus for measuring the blood sugar according to claim 22; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

24. The apparatus of claim 23, wherein the apparatus is configured so that a data of prescription from the hospital staff is transmitted to the blood sugar-measuring apparatus via the two-way communication network.

25. The apparatus of claim 1, wherein the securing means includes a pressing plate for pressing the measurement portion toward the measurement surface.

26. An apparatus for monitoring a blood sugar, comprising: an apparatus for measuring the blood sugar according to claim 25; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

27. The apparatus of claim 26, wherein the apparatus is configured so that a data of prescription from the hospital staff is transmitted to the blood sugar-measuring apparatus via the two-way communication network.

28. The apparatus of claim 1, wherein the securing means is disposed so as to surround the measurement surface and comprising an elastic material so as to be expandable according to the measurement portion.

29. An apparatus for monitoring a blood sugar, comprising: an apparatus for measuring the blood sugar according to claim 28; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

30. The apparatus of claim 29, wherein the apparatus is configured so that a data of prescription from the hospital staff is transmitted to the blood sugar-measuring apparatus via the two-way communication network.

31. The apparatus of claim 1, wherein a surface of the securing means facing the measurement portion is coated with a conductive substance.

32. An apparatus for monitoring a blood sugar, comprising: an apparatus for measuring the blood sugar according to claim 31; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

33. The apparatus of claim 32, wherein the apparatus is configured so that a data of prescription from the hospital staff is transmitted to the blood sugar-measuring apparatus via the two-way communication network.

34. The apparatus of claim 19, wherein the apparatus further comprises an image-recognizing means disposed on the measurement surface for interacting with the control part and identifying the measurement portion of the user.

35. An apparatus for monitoring a blood sugar, comprising: an apparatus for measuring the blood sugar according to claims 34; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

36. The apparatus of claim 35, wherein the apparatus is configured so that a data of prescription from the hospital staff is transmitted to the blood sugar-measuring apparatus via the two-way communication network.

37. The apparatus of claim 34, wherein the apparatus is configured so that the image-recognizing means reads an image of dermatoglyphics of the user and said image is stored in the memory part, and wherein the control part performs an identification of a user and a measurement of blood sugar by using the stored image.

38. An apparatus for monitoring a blood sugar, comprising: an apparatus for measuring the blood sugar according to claim 37; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

39. The apparatus of claim 38, wherein the apparatus is configured so that a data of prescription from the hospital staff is transmitted to the blood sugar-measuring apparatus via the two-way communication network.

40. The apparatus of claim 37, wherein the image recognizing means includes a sensor capable of recognizing a fingerprint, dermatoglyphics or a skin wrinkle pattern of the user.

41. An apparatus for monitoring a blood sugar, comprising: an apparatus for measuring the blood sugar according to claim 40; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

42. The apparatus of claim 41, wherein the apparatus is configured so that a data of prescription from the hospital staff is transmitted to the blood sugar-measuring apparatus via the two-way communication network.

43. The apparatus of claim 19, wherein the displaying means includes: an input part, to which blood sugar data are inputted from the control part; a display part for displaying the inputted blood sugar data; and an output part for externally outputting the blood sugar data.

44. An apparatus for monitoring a blood sugar, comprising: an apparatus for measuring the blood sugar according to claim 43; a computer server for a user, which is connected to the blood sugar-measuring apparatus and to which the measured blood sugar value is inputted; a computer server for the hospital staff, which is connected to the computer server for the user via a two-way communication network and to which the blood sugar value is transmitted; and a terminal connected to the computer server for the hospital staff and being configured so that the transmitted blood sugar value is identified by the hospital staff.

45. The apparatus of claim 44, wherein the apparatus is configured so that a data of prescription from the hospital staff is transmitted to the blood sugar-measuring apparatus via the two-way communication network.
